(19) United States
(12) Patent Application Publication
(10) Pub. No.: US 2008/0020006 A1
(43) Pub. Date: Jan. 24, 2008

(54) Stabilisation Of Colorants In Cosmetic And Dermatological Preparations

(75) Inventor: Valerie Andre, Ludwigshafen (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/629,422

(22) PCT Filed: Jun. 9, 2005
(86) PCT No.: PCT/EP05/06198

§ 371 Date: Dec. 13, 2006

(30) Foreign Application Priority Data
Jun. 15, 2004 (DE) 10 2004 028 629.9

(51) Int. Cl. A61K 8/02; A61K 31/12; A61Q 99/00

(52) U.S. Cl. 424/401; 514/687

(57) ABSTRACT

Methods of stabilizing color in a composition, which methods comprise: (a) providing an amino-substituted hydroxybenzophenone compound corresponding to the general formula (I): 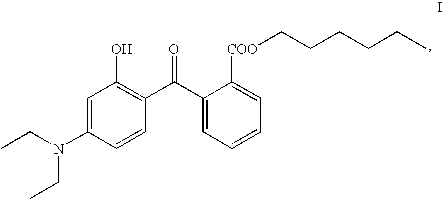 and (b) combining the amino-substituted hydroxybenzophenone compound with a composition to be stabilized selected from the group consisting of cosmetic preparations and dermatological preparations, are described along with compositions containing an organic dye and an amino-substituted hydroxybenzophenone compound corresponding to the general formula (I).

The present invention relates to stabilizing the color of cosmetic and dermatological preparations.

Cosmetic compositions exhibit a certain tendency to decompose during storage. In this connection, it may, for example, be a phase separation in the case of emulsion preparations, changes in the viscosity behavior or changes in the color and odor. The color and odor stability in particular play an important role for cosmetic acceptance by the end consumer. Color and odor of cosmetic preparations can change if the preparations are subjected to elevated temperatures or light, for example in window displays.

In order to reduce the changes caused by light, cosmetic compositions are therefore usually bottled in nontransparent, light-impermeable containers. To improve the photostability, transparent or semitransparent product containers are often manufactured from materials to which UV filter substances, such as, for example, benzotriazole, are added. On the other hand, the cosmetic compositions themselves can comprise UV filter substances.

DE 197 50 906 and DE 197 39 797 disclose, for example, the use of triazine derivatives for stabilizing organic materials against UV light, oxygen and elevated temperatures. EP 0 714 880 relates to bismethylenephenylene derivatives which are not only used as sunscreen factors for protecting the skin and hair, but also serve to improve the storage stability of cosmetic compositions.

Japanese laid-open specifications JP 09078085 A and JP 10237488 A disclose skin and hair cleaning compositions to which complexing agents and antioxidants are added to improve the storage stability at elevated temperature. Stabilizer mixtures of antioxidants and complexing agents are often also used for stabilizing retinoid-containing preparations, as described, for example, in EP 0 440 398, WO 96/07396, EP 0 549 592 and EP 0 586 106.

The current consumer trend is toward cosmetic products in transparent and semitransparent containers. It was therefore the object to develop a stabilizer system which stabilizes cosmetic products in transparent or semitransparent containers against the color and odor changes caused by light. The current compositions are not always sufficiently effective in this regard.

This object was achieved through the use of the amino-substituted hydroxybenzophenone compound of the formula I, 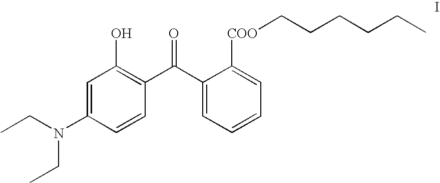
for stabilizing the color of cosmetic and dermatological preparations.

The cosmetic and dermatological preparations suitable for the stabilization within the scope of the present invention preferably comprise one or more organic dyes. The organic dye may be a water-soluble or oil-soluble dye or an insoluble compound obtained by so-called laking of the soluble dyes with metal ions, preferably sodium, calcium or aluminum.

For example, the dyes can be compounds selected from the corresponding positive list of the Cosmetics Directive or the EU list of cosmetic colorants.

In one preferred embodiment of the use according to the invention, the organic dyes are compounds chosen from the group consisting of curcumin (E 100), riboflavin (E 101), lactoflavin (E 101a), tartrazine (E 102), quinoline yellow (E 104), yellow orange S (E 110), cochineal (E 120), azorubin (E 122), amaranth (E 123), cochineal red (E 124), erythrosine (E 127), red 2 G (E 128), allura red AC (E 129), patent blue V (E 131), indigo tin I (E 132), brilliant blue FCF (E 133), chlorophylls (E 140), copper-containing complexes of the chlorophylls (E 141), brilliant acid green (E 142), carotenoids (E 160), 1-carotene (E 160a), annato, bixin, norbixin (E 160b), capsanthin (E 160c), lycopene (E 160d), β-apo-8-carotenal (E 160e), β-apo-8-carotenic ethyl ester (E 160e), xanthophylls (E 161), lutein (E 161b), canthaxanthin (E 161g), beetroot (E 162) and anthocyans (E 163).

Particularly preferred dyes are tartrazine (E 102), quinoline yellow (E 104), yellow orange S (E 110), cochineal red (E 124), indigo tin I (E 132).

According to the invention, the compound of the formula I is used in concentrations of from 0.01 to 5% by weight, preferably from 0.05 to 3% by weight, particularly preferably in concentrations of from 0.08 to 2% by weight, based on the total amount of cosmetic or dermatological preparation.

In a further preferred embodiment of the use according to the invention, the amino-substituted hydroxybenzophenone compound of the formula I is used in a mixture together with the p-methoxycinnamic ester of the formula II.

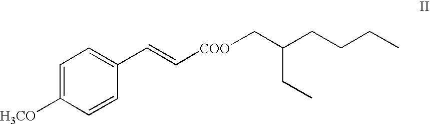

The composition of the mixture consists preferably of 30 to 70% by weight, particularly preferably 30 to 50% by weight, very particularly preferably 35 to 45% by weight, of the compound I and 70 to 30% by weight, particularly preferably 70 to 50% by weight, very particularly preferably 65 to 55% by weight, of the compound II.

The invention also provides cosmetic or dermatological preparations comprising at least one organic dye and the amino-substituted hydroxybenzophenone compound of the formula I.

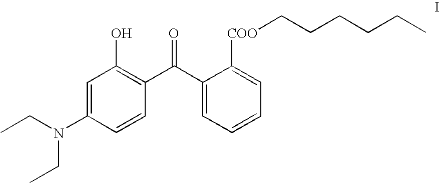

Preferred organic dyes of the cosmetic or dermatological preparations are compounds chosen from the group consisting of curcumin (E 100), riboflavin (E 101), lactoflavin (E 101a), tartrazine (E 102), quinoline yellow (E 104), yellow orange S (E 110), cochineal (E 120), azorubin (E 122), amaranth (E 123), cochineal red (E 124), erythrosine (E 127), red 2 G (E 128), allura red AC (E 129), patent blue V (E 131), indigo tin I (E 132), brilliant blue FCF (E 133), chlorophylls (E 140), copper-containing complexes of the chlorophylls (E 141), brilliant acid green (E 142), carotenoids (E 160), β-carotene (E 160a), annato, bixin, norbixin (E 160b), capsanthin (E 160c), lycopene (E 160d), β-apo-8-carotenal (E 160e), β-apo-8-carotenic ethyl ester (E 160e), xanthophylls (E 161), lutein (E 161b), canthaxanthin (E 161g), beetroot (E 162) and anthocyans (E 163).

Particularly preferred dyes are tartrazine (E 102), quinoline yellow (E 104), yellow orange S (E 110), cochineal red (E 124), indigo tin I (E 132).

A further preferred form of the preparations additionally comprises the p-methoxycinnamic ester of the formula II.

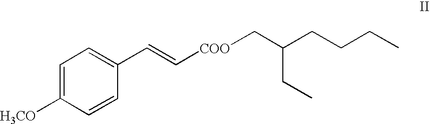

The preparations may be cosmetic cleaning products such as foam baths, shower baths, cleaning foams, shampoos, haircare products, such as hair gels and hair lotions, and products for bodycare and skincare, such as creams, lotions, deodorants etc. The products can also be formulated on an aqueous, aqueous-alcoholic or aqueous-glycolic basis, and comprise, for example, toilet waters, face tonics and aftershaves. In principle, there are no restrictions here.

The cosmetic preparations preferably comprise at least one surfactant from the group of anionic surfactants, nonionic surfactants, amphoteric surfactants, zwitterionic surfactants and/or cationic surfactants.

The surfactants are present in amounts of from 5 to 40% by weight, preferably 5 to 20% by weight, in each case based on the weight of the cosmetic preparation.

Suitable anionic surfactants in compositions according to the invention are all anionic surface-active substances suitable for use on the human body. These are characterized by a water-solubilizing, anionic group, such as, for example, a carboxylate, sulfate, sulfonate or phosphate group, and a lipophilic alkyl group having about 10 to 22 carbon atoms. Additionally, glycol groups or polyglycol ether groups, ester groups, ether groups and amide groups, and hydroxyl groups may be present in the molecule.

Examples of suitable anionic surfactants are, in each case in the form of the sodium, potassium, magnesium and ammonium, and also the mono-, di- and trialkanolammonium salts having 2 or 3 carbon atoms in the alkanol group,
linear fatty acids having 10 to 22 carbon atoms (soaps),
ether carboxylic acids of the formula R¹—O—(CH₂—CH₂O)ₓ—CH₂—COOH, in which R¹ is linear alkyl group having 10 to 22 carbon atoms and x=0 or 1 to 16,
amide ether carboxylates of the formula [R²—NH(—CH₂—CH₂—O)ₙ—CH₂—COO]ₘZ, in which R² is a linear or branched, saturated or unsaturated acyl radical having 2 to 29 carbon atoms, n is integers from 1 to 10, m is numbers 1 or 2 and Z is a cation from the group of alkali metals or alkaline earth metals,
acyl sarcosides having 10 to 18 carbon atoms in the acyl group,
acyl taurides having 10 to 18 carbon atoms in the acyl group,
acyl isethionates having 10 to 18 carbon atoms in the acyl group,
sulfosuccinic mono- and dialkyl esters having 8 to 18 carbon atoms in the alkyl group and sulfosuccinic monoalkyl polyoxyethyl esters having 8 to 18 carbon atoms in the alkyl group and 1 to 6 oxyethyl groups,
linear alkanesulfonates having 12 to 18 carbon atoms,
linear alpha-olefinsulfonates having 12 to 18 carbon atoms,
alpha-sulfofatty acid methyl esters of fatty acids having 12 to 18 carbon atoms,
alkyl sulfates and alkyl polyglycol ether sulfates of the formula R³—O(—CH₂—CH₂O)ₓ—SO₃H, in which R³ is a preferably linear alkyl group having 10 to 18 carbon atoms and x=0 or 1 to 12,
mixtures of surface-active hydroxysulfonates as in DE-A-37 25 030,
sulfated hydroxyalkyl polyethylene and/or hydroxyalkylene propylene glycol ethers as in DE-A-37 23 354,
sulfonates of unsaturated fatty acids having 12 to 24 carbon atoms and 1 to 6 double bonds as in DE-A-39 26 344,
esters of tartaric acid and citric acid with alcohols, which constitute addition products of about 2-15 molecules of ethylene oxide and/or propylene oxide onto fatty alcohols having 8 to 22 carbon atoms,
coconut monoglyceride sulfates.

Preferred anionic surfactants are alkyl sulfates, alkyl polyglycol ether sulfates and ether carboxylic acids having 10 to 18 carbon atoms in the alkyl group and up to 12 glycol ether groups in the molecule, and sulfosuccinic mono- and dialkyl esters having 8 to 18 carbon atoms in the alkyl group and sulfosuccinic monoalkyl polyoxyethyl esters having 8 to 18 carbon atoms in the alkyl group and 1 to 6 oxyethyl groups.

Nonionogenic surfactants comprise, as hydrophilic group, e.g. a polyol group, a polyalkylene glycol ether group or a combination of polyol group and polyglycol ether group. Such compounds are, for example,
addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C₁₂-C₂₂ fatty acid mono- and diesters of addition products of from 1 to 30 mol of ethylene oxide onto glycerol,
C₈-C₂₂-alkyl mono- and oligoglycosides and ethoxylated analogs thereof, and
addition products of from 5 to 60 mol of ethylene oxide onto castor oil and hydrogenated castor oil.

Preferred nonionic surfactants are alkyl polyglycosides of the general formula R⁴O—(S)ₓ. These aid the mildness of the preparations according to the invention, have a thickening effect and contribute to improved solubilization of the fatty acid partial glycerides. They are characterized by the following parameters:

The alkyl radical R⁴ comprises 6 to 22 carbon atoms and may either be linear or branched. Preference is given to primary linear and methyl-branched in the 2-position aliphatic radicals. Such alkyl radicals are, for example, 1-octyl, 1-decyl, 1-lauryl, 1-myristyl, 1-cetyl and 1-stearyl. Particular preference is given to 1-octyl, 1-decyl, 1-lauryl, 1-myristyl.

When using so-called oxo alcohols as starting materials, compounds with an odd number of carbon atoms in the alkyl chain predominate.

The alkyl polyglycosides which can be used according to the invention can, for example, comprise only one specific alkyl radical R⁴. Usually, these compounds, however, are prepared starting from natural fats and oils or mineral oils. In this case, the alkyl radicals R⁴ present are mixtures corresponding to the starting compounds and/or corresponding to the particular work-up of these compounds.

Particular preference is given to those alkyl polyglycosides in which R⁴ consists
essentially of C₈- and C₁₀-alkyl groups,
essentially of C₁₂- and C₁₄-alkyl groups,
essentially of C₈- to C₁₆-alkyl groups or
essentially of C₁₂- to C₁₆-alkyl groups.

The sugar building block S which may be used is any desired mono- or oligosaccharide. Usually, sugars with 5 or 6 carbon atoms, and the corresponding oligosaccharides are used. Such sugars are, for example, glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is particularly preferred.

The alkyl polyglycosides which can be used according to the invention comprise, on average, 1.1 to 5 sugar units. Alkyl polyglycosides with x values of from 1.1 to 1.6 are preferred. Very particular preference is given to alkyl glycosides in which x is 1.1 to 1.4.

Besides their surfactant effect, the alkyl glycosides can also serve to improve the fixing of the scent components on the hair and the skin.

The alkoxylated homologs of the specified alkyl polyglycosides can also be used according to the invention. These homologs can comprise, on average, up to 10 ethylene oxide and/or propylene oxide units per alkyl glycoside unit.

The compounds with alkyl groups used as surfactants may each be single substances. However, it is usually preferred, when producing these substances, to start from native vegetable or animal raw materials, giving rise to mixtures of substances with varying alkyl chain lengths depending on the particular raw material.

In the case of the surfactants which constitute addition products of ethylene oxide and/or propylene oxide onto fatty alcohols or derivatives of these addition products, it is possible to use either products with a “normal” homolog distribution, or those with a narrowed homolog distribution. “Normal” homolog distribution is understood here as meaning mixtures of homologs which are obtained in the reaction of fatty alcohol and alkylene oxide using alkali metals, alkali metal hydroxides or alkali metal alkoxides as catalysts. Narrowed homolog distributions, on the other hand, are obtained if, for example, hydrotalcites, alkaline earth metal salts of ether carboxylic acids, alkaline earth metal oxides, hydroxides or alkoxides are used as catalysts. The use of products with a narrowed homolog distribution may be preferred.

In addition, zwitterionic surfactants can be used, particularly as cosurfactants. Zwitterionic surfactants is the term used for those surface-active compounds which carry at least one quaternary ammonium group and at least one —COO⁻ or —SO₃⁻ group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazoline having in each case 8 to 18 carbon atoms in the alkyl or acyl group, and also cocoacylaminoethyl hydroxyethylcarboxymethylglycinate. A preferred zwitterionic surfactant is the fatty acid amide derivative known under the INCI name Cocamidopropyl Betaine.

Likewise particularly suitable as cosurfactants are ampholytic surfactants. Ampholytic surfactants are understood as meaning those surface-active compounds which, apart from a C₈-C₁₈-alkyl or acyl group in the molecule, comprise at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, -hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂₋₁₈-acylsarcosine.

Examples of the cationic surfactants which are preferably used in hair-treatment compositions are, in particular, quaternary ammonium compounds. Preference is given to ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride.

In addition, the very readily biodegradable quaternary ester compounds, such as, for example, the dialkylammonium methosulfates and methylhydroxyalkyldialkoyloxy-alkylammonium methosulfates sold under the trade name Stepantex® and the corresponding products of the Dehyquart® series can be used as cationic surfactants.

Further cationic surfactants which can be used according to the invention are the quaternized protein hydrolyzates.

In one preferred embodiment, it is a cleaning foam with stabilizer according to the invention which comprises, as surfactant components, a combination of a sulfosuccinic monoester salt, an ampho or betaine surfactant and an alkyl polyglycoside.

In one particularly preferred embodiment of the invention, it is a foam cleaning product for bodycare and facecare, consisting of a surfactant-containing cleaner comprising the stabilizer.

Apart from the stabilizer, the cosmetic product also comprises, in one preferred embodiment, at least one care active ingredient chosen from the group of vitamins, provitamins or vitamin precursors.

These are present in the compositions according to the invention in an amount of 0.1-10% by weight, preferably 0.2-5% by weight and in particular 0.5-1% by weight, in each case based on the weight of the cosmetic preparation. Here, preference is given according to the invention to those vitamins, provitamins and vitamin precursors which are usually assigned to the groups A, B, C, F and H.

The group of substances referred to as vitamin A includes retinol (vitamin Al) and 3,4-didehydroretinol (vitamin A₂). β-Carotene is the provitamin of retinol. According to the invention, suitable vitamin A components are, for example, vitamin A acid and esters thereof, Vitamin A aldehyde and vitamin A alcohol, and esters thereof, such as the palmitate and the acetate.

The vitamin B group or vitamin B complex include, inter alia,

vitamin B₁ (thiamine)

vitamin B₂ (riboflavin)

Vitamin B₃. The compounds nicotinic acid and nicotinamide (niacinamide) often go under this name, of which nicotinamide in particular is preferred according to the invention.

Vitamin B₅ (pantothenic acid and panthenol). Within this group, preference is given to using panthenol. Derivatives of panthenol which can be used according to the invention are, in particular, the esters and ethers of panthenol, cationically derivatized panthenols and pantolactone.

Vitamin B₆ (pyridoxine and pyridoxamine and pyridoxal).

Also suitable according to the invention are vitamin C (ascorbic acid) and esters thereof, in particular ascorbyl palmitate.

Vitamin F. The term “vitamin F” is usually understood as meaning essential fatty acids, in particular linoleic acid, linolenic acid and arachidonic acid.

Vitamin H. Vitamin H is the name for the compound (3aS,4S,6aR)-2-oxohexahydrothienol-[3,4-d]-imidazole-4-valeric acid, for which, however, in the meantime the trivial name biotin has caught on.

In a further preferred embodiment, the cosmetic composition of the product according to the invention comprises at least one plant extract or a distillate of plant constituents.

Plant extracts and plant distillates often increase the other active ingredient properties of the composition. According to the invention, the plant extracts and plant distillates can either be used in pure form or in dilute form. The compositions according to the invention can also comprise mixtures of two or more different plant extracts and plant distillates. The plant extracts and plant distillates are usually present in an amount of from 0.01-5% by weight, preferably 0.1-3% by weight and in particular 0.1-2% by weight, of active substance, in each case based on the weight of the cosmetic preparation.

These extracts and distillates are usually obtained by extraction of the whole plant or by steam distillation. In individual cases, however, it may also be preferred to produce the extracts and distillates exclusively from flowers and/or leaves of the plant.

With regard to the plant extracts which can be used according to the invention, reference is made in particular to the extracts which are listed in the table starting on page 44 of the 3rd edition of the introduction to the Ingredients Declaration of Cosmetic Compositions, published by the Industrieverband Körperpflege-und Waschmittel e.V. (JKW), Frankfurt.

According to the invention, the extracts and distillates from green tea, oak bark, stinging nettle, hamamelis, hops, camomile, burdock, horsetail, hawthorn, linden blossom, almond, aloe vera, fir needle, horse chestnut, sandalwood, juniper, coconut, mango, apricot, lime, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are primarily preferred.

According to the invention, preference is given to almond extracts and distillates from hamamelis and sage.

Extractants for producing said plant extracts which can be used are water, alcohols, and mixtures thereof. Among the alcohols here, lower alcohols such as ethanol and isopropanol, but in particular polyhydric alcohols, such as ethylene glycol and propylene glycol, either as the sole extractant or in a mixture with water, are preferred here.

Plant extracts based on water/propylene glycol in the ratio 1:10 to 10:1 have proven to be particularly suitable.

Preferably, the compositions according to the invention further comprise at least one organic thickener. Such thickeners are, for example, thickeners such as agar-agar, guar gum, alginates, cellulose ethers, gelatin, pectins and/or xanthan gum. Ethoxylated fatty alcohols, in particular those with a narrowed homolog distribution, as are marketed, for example, as commercial product under the name Arlypon® F (Henkel), alkoxylated methyl glucoside esters, such as the commercial product Glutamate® DOE 120 (Amerchol), and ethoxylated propylene glycol esters, such as the commercial product Antil® 141 (Goldschmidt) may be preferred organic thickeners.

Silicone oils and silicone gums suitable as conditioning active ingredients are, in particular, dialkyl- and alkylarylsiloxanes, such as, for example, dimethylpolysiloxane and methylphenylpolysiloxane, and alkoxylated and quaternized analogs thereof. Examples of such silicones are the products sold by Dow Corning under the names DC 190, DC 200 and DC 1401, and also the commercial product Fancorsil® LIM-1. A suitable anionic silicone oil is the product Dow Corning® 1784.

Vegetable oils and waxes preferably present are evening primrose oil, jojoba oil, sunflower oil, orange oil, almond oil, wheat germ oil and peach kernel oil. Evening primrose oil is particularly preferred.

Especially for the formulation of very mild cosmetic preparations, it has also proven to be advantageous if the amount of dissolved inorganic salts is limited to less than 2% by weight, in particular less than 0.5% by weight. Here, it should also be taken into consideration that such salts are not only added, for example, to adjust the viscosity, but can also be introduced via other active ingredients, in particular surfactants.

Further customary constituents for the compositions according to the invention are:
nonionic polymers, such as, for example, vinylpyrrolidone/vinyl acrylate copolymers, polyvinylpyrrolidone and vinylpyrrolidone/vinyl acetate copolymers,
anionic polymers, such as polyacrylic and polymethacrylic acids, salts thereof, copolymers thereof with acrylic and methacrylic esters and acryl- and methacrylamides and derivatives thereof which are obtained by crosslinking with polyfunctional agents,
polyoxycarboxylic acids, such as polyketo- and polyaldehydrocarboxylic acids and salts thereof, and polymers and copolymers of crotonic acid with esters and amides of acrylic acid and of methacrylic acid, such as vinyl acetate-crotonic acid and vinyl acetate-vinyl propionate-crotonic acid copolymers,
structurants, such as glucose and maleic acid,
hair-conditioning compounds, such as phospholipids, for example soya lecithin, egg lecithin and cephalins,
perfume oils, in particular those with the scent note of a fruit, such as, for example, of apple, pear, strawberry, peach, apricot, pineapple, banana, cherry, kiwi, mango, coconut, almond, grapefruit, maracuja, mandarin and melon, or the scent note of a luxury product, such as, for example, of tobacco, cola, chewing gum, guarana, chocolate, cocoa, vanilla, sarsaparilla, peppermint and rum.
Solubility promoters, such as ethanol, isopropanol, ethylene glycol, propylene glycol, glycerol, diethylene glycol and ethoxylated triglycerides,
dimethyl isosorbide and cyclodextrins,
dyes,
photoprotective agents,
fats and waxes, such as spermaceti, beeswax, montan wax, paraffins, esters,
antidandruff active ingredients, such as climbazole, piroctone olamine and zinc omadine,
active ingredients, such as bisabolol and allantoin,
consistency regulators, such as sugar esters, polyol esters or polyol alkyl ethers,
glycerides and fatty alcohols,
fatty acid alkanolamides,
swelling and penetration substances, such as PCA, glycerol, propylene glycol monoethyl ether, carbonates, hydrogencarbonates, guanidines, ureas, and primary, secondary and tertiary phosphates,
opacifiers, such as latex or styrene/acrylamide copolymers,
pearlizing agents, such as ethylene glycol mono- and distearate or PEG-3 distearate,
direct dyes
antioxidants,
preservatives,
propellants, such as propane/butane mixtures, N20, dimethyl ether, CO2 and air, and
bitter substances, such as, for example, denatonium benzoate,

The examples below are intended to illustrate the subject matter of the invention in more detail without limiting it thereto.

### EXAMPLES OF COSMETIC FORMULATIONS

### Example 1

### Hair Tonic

| % | Raw material | Batch (400 g) |
|---|---|---|
| Phase A | | |
| 40.0 | Ethanol abs. | Alcohol |
| 2.0 | Cremophor CO 40 | PEG-40 Hydrogenated Castor Oil |
| 0.1 | Bisabolol, rac. | Bisabolol |
| 0.1 | Compound I | |
| Phase B | | |
| 2.0 | Luviquat FC 550 | Polyquaternium-16 |
| 1.0 | Dye solution (quinoline | |
| | yellow) 1% in water | |
| 54.8 | Water, demin. | Water |

### Example 2

### Shampoo

| % | | |
|---|---|---|
| Phase A | Raw material | Batch (400 g) |
| 40.0 | Texapon NSO | Sodium Laureth Sulfate |
| 5.0 | Tego Betain HS | Cocamidopropyl Betaine, |
| | | Glyceryl Laurate |
| 1.0 | Comperlan KD | Cocamide DEA |
| 5.0 | Luviquat FC 550 | Polyquaternium-16 |
| 1.0 | Luviquat Mono CP | Hydroxyethyl Cetyldimonium |
| | | Phosphate |
| 0.1 | Euxyl K 100 | Benzyl Alcohol, |
| | | Methylchloroisothiazolinone, |
| | | Methylisothiazolinone |
| 0.1 | Compound I | |
| 0.1 | Edeta BD | Disodium EDTA |
| 1.0 | Dye solution (quinoline | |
| | yellow) 1% in water | |
| 54.8 | Water, demin. | Water |

### Example 3

### Hair Tonic

| % | Raw material | Batch (400 g) |
|---|---|---|
| Phase A | | |
| 40.0 | Ethanol abs. | Alcohol |
| 2.0 | Cremophor CO 40 | PEG-40 Hydrogenated Castor Oil |
| 0.1 | Bisabolol, rac. | Bisabolol |
| 0.1 | Mixture of compound I | |
| | (35% by wt.) and | |
| | compound II (65% by wt.) | |
| Phase B | | |
| 2.0 | Luviquat FC 550 | Polyquaternium-16 |
| 1.0 | Dye solution (quinoline | |
| | yellow) 1% in water | |
| 54.8 | Water, demin. | Water |

### Example 4

### Shampoo

| % | | |
|---|---|---|
| Phase A | Raw material | Batch (400 g) |
| 40.0 | Texapon NSO | Sodium Laureth Sulfate |
| 5.0 | Tego Betain HS | Cocamidopropyl Betaine, |
| | | Glyceryl Laurate |
| 1.0 | Comperlan KD | Cocamide DEA |
| 5.0 | Luviquat FC 550 | Polyquaternium-16 |
| 1.0 | Luviquat Mono CP | Hydroxyethyl Cetyldimonium |
| | | Phosphate |
| 0.1 | Euxyl K 100 | Benzyl Alcohol, |
| | | Methylchloroisothiazolinone, |
| | | Methylisothiazolinone |
| 0.1 | Mixture of compound I | |
| | (35% by wt.) and | |
| | compound II (65% by wt.) | |
| 0.1 | Edeta BD | Disodium EDTA |
| 1.0 | Dye solution (quinoline | |
| | yellow) 1% in water | |
| 54.8 | Water, demin. | Water |

1-11. (canceled)

12. A method of stabilizing color in a composition, the method comprising:
(a) providing an amino-substituted hydroxybenzophenone compound corresponding to the general formula (I): 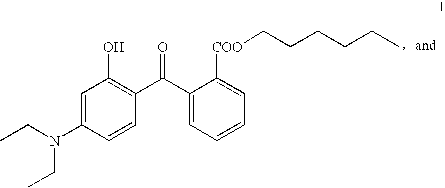
(b) combining the amino-substituted hydroxybenzophenone compound with a composition to be stabilized selected from the group consisting of cosmetic preparations and dermatological preparations.

13. The method according to claim 12, wherein the composition to be stabilized comprises an organic dye.

14. The method according to claim 13, wherein the organic dye comprises a component selected from the group consisting of water-soluble dyes, oil-soluble dyes, water-insoluble colored lakes, and mixtures thereof.

15. The method according to claim 13, wherein the organic dye comprises a component selected from the group consisting of curcumin E100, riboflavin E 101, lactoflavin E101a, tartrazine E102, quinoline yellow E104, yellow orange S E110, cochineal E120, azorubin E122, amaranth E123, cochineal red E124, erythrosine E127, red 2 G E128, allura red AC E129, patent blue V E131, indigo tin I E132, brilliant blue FCF E133, chlorophylls E140, copper-containing complexes of the chlorophylls E141, brilliant acid green E142, carotenoids E160, β-carotene E160a, annato, bixin, norbixin E160b, capsanthin E160c, lycopene E160d, β-apo-8-carotenal E160e, β-apo-8-carotenic ethyl ester E160e, xanthophylls E161, lutein E161b, canthaxanthin E161g, beetroot E162, anthocyans E163, and mixtures thereof.

16. The method according to claim 12, wherein the amino-substituted hydroxybenzophenone compound is combined with the composition in an amount of 0.01 to 5% by weight, based on the total weight of the composition.

17. The method according to claim 14, wherein the amino-substituted hydroxybenzophenone compound is combined with the composition in an amount of 0.01 to 5% by weight, based on the total weight of the composition.

18. The method according to claim 15, wherein the amino-substituted hydroxybenzophenone compound is combined with the composition in an amount of 0.01 to 5% by weight, based on the total weight of the composition.

19. The method according to claim 12, wherein the amino-substituted hydroxybenzophenone compound is provided as a mixture with a p-methoxycinnamic ester corresponding to the general formula II: 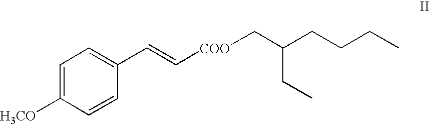
and the mixture is combined with the composition.

20. The method according to claim 14, wherein the amino-substituted hydroxybenzophenone compound is provided as a mixture with a p-methoxycinnamic ester corresponding to the general formula II: 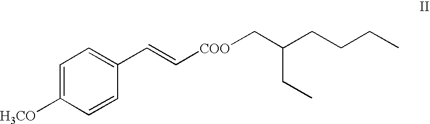
and the mixture is combined with the composition.

21. The method according to claim 16, wherein the amino-substituted hydroxybenzophenone compound is provided as a mixture with a p-methoxycinnamic ester corresponding to the general formula II: 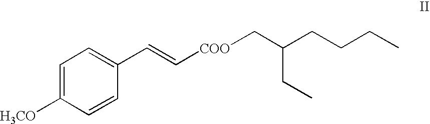
and the mixture is combined with the composition.

22. The method according to claim 19, wherein the mixture comprises 30 to 70% by weight of the amino-substituted hydroxybenzophenone compound and 70 to 30% by weight of the p-methoxycinnamic ester, based on the weight of the mixture.

23. A composition comprising an organic dye and an amino-substituted hydroxybenzophenone compound corresponding to the general formula (I):

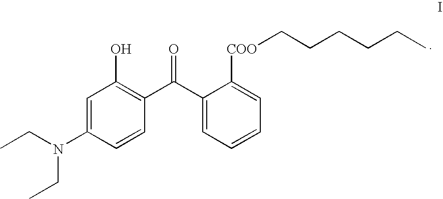

24. The composition according to claim 23, wherein the organic dye comprises a component selected from the group consisting of curcumin E100, riboflavin E 101, lactoflavin E101a, tartrazine E102, quinoline yellow E104, yellow orange S E110, cochineal E120, azorubin E122, amaranth E123, cochineal red E124, erythrosine E127, red 2 G E128, allura red AC E129, patent blue V E131, indigo tin I E132, brilliant blue FCF E133, chlorophylls E140, copper-containing complexes of the chlorophylls E141, brilliant acid green E142, carotenoids E160, β-carotene E160a, annato, bixin, norbixin E160b, capsanthin E160c, lycopene E160d, β-apo-8-carotenal E160e, β-apo-8-carotenic ethyl ester E160e, xanthophylls E161, lutein E161b, canthaxanthin E161g, beetroot E162, anthocyans E163, and mixtures thereof.

25. The composition according to claim 23, further comprising a p-methoxycinnamic ester corresponding to the general formula II:

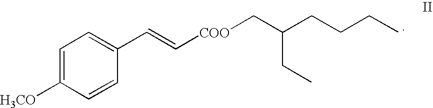

26. The composition according to claim 24, further comprising a p-methoxycinnamic ester corresponding to the general formula II:

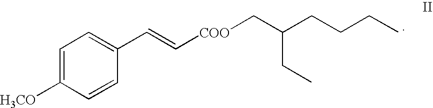

27. The composition according to claim 23, wherein the amino-substituted hydroxybenzophenone compound is present in an amount of 0.01 to 5% by weight, based on the total weight of the composition.

28. The composition according to claim 25, wherein the amino-substituted hydroxybenzophenone compound is present in an amount of 30 to 70% by weight and the p-methoxycinnamic ester is present in an amount of 70 to 30% by weight, based on the combined total weight of the amino-substituted hydroxybenzophenone compound and the p-methoxycinnamic ester.

29. The composition according to claim 23, wherein the composition is a cosmetic or dermatological preparation selected from the group consisting of shampoos, hair gels, hair lotions, skin creams, skin lotions, deodorants, toilet waters, face tonics, aftershaves and combinations thereof.

30. The composition according to claim 25, wherein the composition is a cosmetic or dermatological preparation selected from the group consisting of shampoos, hair gels, hair lotions, skin creams, skin lotions, deodorants, toilet waters, face tonics, aftershaves and combinations thereof.

31. The composition according to claim 27, wherein the composition is a cosmetic or dermatological preparation selected from the group consisting of shampoos, hair gels, hair lotions, skin creams, skin lotions, deodorants, toilet waters, face tonics, aftershaves and combinations thereof.
